(19) United States
(12) Patent Application Publication
(10) Pub. No.: US 2014/0017135 A1
(43) Pub. Date: Jan. 16, 2014

(54) Systems and Methods for Disinfecting Air On Board A Passenger Transport Vehicle

(71) Applicant: MAG AEROSPACE INDUSTRIES, INC., Carson, CA (US)

(72) Inventors: Razmik B. Boodaghians, Glendale, CA (US); Kevin Huang, Los Angeles, CA (US); Christoph Goeschel, Seattle, WA (US); Christina Ortolan, Long Beach, CA (US); Vikram Chauhan, Redondo Beach, CA (US)

(21) Appl. No.: 13/942,821

(22) Filed: Jul. 16, 2013

Related U.S. Application Data
(60) Provisional application No. 61/671,902, filed on Jul. 16, 2012.

(51) Int. Cl. A61L 9/20

(52) U.S. Cl. CPC A61L 9/205
USPC 422/121

(57) ABSTRACT

Embodiments of the present invention relate generally to air sanitation and disinfection, and particularly to air sanitation and disinfection for aerospace applications.

### CROSS REFERENCE TO RELATED APPLICATIONS

This application claims the benefit of U.S. Provisional Application Ser. No. 61/671,902, filed Jul. 16, 2013, titled “Systems and Methods for Disinfecting Air Using Ultraviolet Light and Oxidative/Reactive Molecular Species,” the entire contents of which are hereby incorporated by reference.

### FIELD OF THE INVENTION

Embodiments of the present invention relate generally to air sanitation and disinfection, and particularly to air sanitation and disinfection for aerospace applications.

### BACKGROUND

Commercial vehicles typically have the capacity to carry dozens to hundreds of people per trip. With such large numbers of people in a confined space, there is a risk of propogation of bacteria or other pathogens, which can negatively affect passengers and/or equipment. Thus, on aircraft and other passenger transportation vehicles, there is a need for cabin air purification, as well as a need for sanitation of the oxygen supply consumed into certain fuel cell types, such as proton exchange membrane fuel cells (PEMFCs). (The use of fuel cells on aircraft and other vehicles has recently been explored as a viable way to power certain aircraft electronics, as well as delivering fuel cell by-products for other uses, such as heat for wing de-icing, oxygen depleted air for fuel tank inerting, water for various needs on-board, and other as described in the current assignee's pending patent applications relating to these and other various fuel cell uses on-board vehicles.)

First, cabin air can become contaminated by chemicals, odors, pathogens, molds, and volatile organic compounds (VOCs). Each of these is potentially dangerous to the people on-board aircraft, and there is an increased risk of spreading sicknesses in the close quarters of the cabin. There are thus needs for purifying cabin air. Second, if pathogens propagate into the fuel cell system, biofilm or other biological degradation can occur, resulting in reduction of performance or even failure of the system. This problem may also be exacerbated in commercial aircraft, which commonly undergo numerous and/or extended intervals of non-operation for maintenance or other servicing between trips, resulting in significant spans of time in which pathogen growth can go unchecked. As such, systems that may be implemented to counteract the spread of bacteria and other pathogens are desirable for safeguarding the health of passengers and/or the operational health of equipment aboard the craft.

Regarding the purification of cabin air, this air can be purified using a simple UV source or a UV source in conjunction with a material which produces oxidative species when exposed to ultraviolet light. Oxidative species are capable of deactivating pathogens in air on contact, including bacteria, viruses, and volatile organic compounds (VOCs). Another air purification system is a High Efficiency Particulate Air (HEPA) Filter; however, this filter is only able to eliminate particulates. It does not address any other types of contaminants.

### BRIEF SUMMARY

Applicants have thus designed an air cleaning device that can purify the cabin air by re-circulating it though an air cleansing module. The air cleansing module described herein is able to sanitize the cabin air, which is not done properly by existing technologies. Accordingly, this new air cleaning device is able to eliminate a more diverse group of contaminants from the air than current air filtration systems found in aircraft cabins. Further, this new technology does not use chemicals or any toxic substances to sanitize the air, which is in the best interest of the passengers on the plane.

This invention is also able to provide valuable air purification to fuel cells, such as proton-exchange membrane fuel cells (PEMFCs). PEMFCs are able to produce electricity when provided with hydrogen and oxygen. However, this same reaction also produces water as a byproduct, which can then be collected and used for various purposes, including potable water. If drinking water can be formed on-board an aircraft using a hydrogen source, such as a tank of hydrogen, and an oxygen source, such as cabin air, then drinking water has the potential to be contaminated with multiple different types of pathogens, odors, molds, and VOCs from cabin air if it is used directly. Since oxygen from the cabin air is a fundamental part of the reactions to produce water, it is important to cleanse it so that the water produced can be used for drinking and/or washing. It is also important to deliver scrubbed oxygen-rich air to the fuel cell in order to ensure that contaminants from the air do not become entrapped in the fuel cell. In order to solve this problem, an air purifying system can be implemented to purify the air that enters the fuel cell, and consequently protect the resultant water production method from any contaminants.

### BRIEF DESCRIPTION OF THE DRAWINGS

FIG. 1 shows a side perspective view of one embodiment of an air cleansing module system.

FIG. 2 shows a top cross-sectional view of an air cleansing module.

FIG. 3 shows a top cross-sectional view of an alternate air cleansing module.

FIG. 4 shows a perspective view of one embodiment of an LED UV light source.

FIG. 5A shows a top cross-sectional view of one embodiment of a reaction chamber having LED components and reflectors.

FIG. 5B shows a side perspective view of an alternate reaction chamber with an LED array.

FIG. 6 shows a perspective exploded view of the air cleansing module system of FIG. 1.

FIG. 7 shows a top and perspective cut away view of one embodiment of a lower cap for use in connection with a modular water treatment system.

FIG. 8 shows a top cross sectional view of a water treatment system having a tripod system to secure and hold a UV bulb.

FIG. 9 shows a side sectional view of the air cleansing module system of FIG. 8.

FIG. 10 shows a side perspective view of an air duct with an air cleansing module incorporated there.

FIG. 11 shows a schematic of air flow treatment through an air cleansing module before delivery to a fuel cell.

### DETAILED DESCRIPTION

The air cleansing module described herein may be used as part of a complete aircraft system to be used in an air filtration and sanitation capacity for cabins and/or fuel cells. The air cleansing module modifies existing aircraft air cleaning technology by containing oxidative species within a reaction chamber and/or dispersing the oxidative species into the atmosphere in question such as the aircraft cabin or PEMFC air source. Furthermore, prior art air purification generally uses UV lamps, but the present system uses a UV LED array, which has the capacity to yield a finely tunable spectrum of UV wavelengths to target a wider range of contaminant species. It also provides a beneficial reacting coating on the surface of the module. The air cleansing module also cleanses air without the use of harmful chemicals and toxins making it a safe alternative to many air sanitation products.

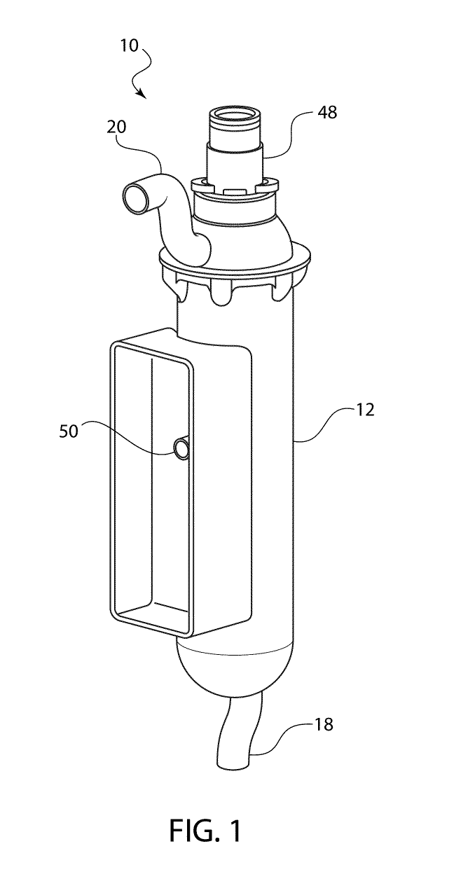

Certain embodiments described herein are adaptations made to current Monogram Systems technology that is used to treat water. Details of the water treatment system are provided in U.S. Publication No. 2012/0051977, the entire contents of which are incorporated herein by reference. The adaptations and modification provided herein allow the system to be used for air sanitation, cleansing, and treatment as well. FIG. 1 shows one embodiment of an air treatment system 10 for aircraft cabins and/or proton exchange membrane fuel cells. In the embodiment shown, air enters through inlet 18 and exits from outlet 20. This embodiment incorporates an ultraviolet (UV) source with a photo-catalytic oxidative (PCO) coating on the inner reaction chamber surface. This PCO coating interacts with the UV light to create radical oxidative species that are able to destroy pathogens adsorbed to the inner surface of the reaction chamber, pathogens resident in the volume of the reaction chamber, and pathogens moving in the air flow of the device. The UV light interacts with the photo-catalyst, so the UV source and the photo-catalyst are placed in close proximity to one another. The PCO coating can be made from multiple different photo-catalytic surfaces. The most commonly used and effective PCO material is titanium-dioxide (TiO₂). TiO₂ has been shown to be non-toxic and a powerful source of hydroxyl and superoxide radicals. When these radicals are created, they react directly with pollutants in the air to destroy them.

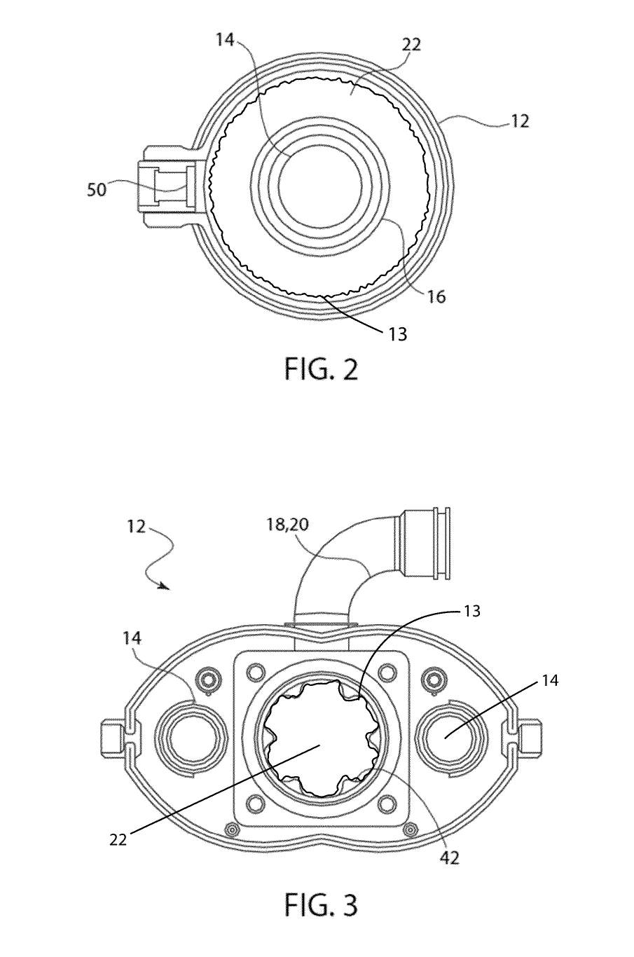

In the view of FIG. 2, the PCO coating 13 is applied to the inner surface of the reaction chamber 12. This coating can be applied either directly to this inner surface, or to protrusions 42 from the inner surface, as shown in FIG. 3. Such protrusions 42 are used to induce turbulent flow of air and increase surface area, and may be referred to as fins 42. Turbulence inducing fins 42, which are positioned in the interior of air flow space 22, are intended to induce turbulence and agitate air in the system. The quartz tube or other sheath of the UV lamp may rest on these fins or teeth for support as well.

In an alternate embodiment, the PCO coating is not used directly. Instead, a photo-catalyst coating of TiO2 or equivalent with additional doping with silver, copper, and rhodium is used. In this embodiment, the system will eject radical species of hydroxyl, super-oxide, hydrogen peroxide, and ozone into the area of interest, which can render the system potentially more active when compared to the direct PCO system, as the radical species attack pollutants at the point of interest, such as the aircraft cabin or PEMFC air source, rather than in a reaction chamber. In one embodiment, the coating reacts with the airstream to release reactive species into the target atmosphere to perform the reactions therein. In another embodiment, the device may be located at the central circulation point or at an air distribution point.

The structural features shown and described are all shown and described in co-pending application Ser. No. 12/872,420, Publication No. 2012/0051977, the entire contents of which are incorporated herein. The present inventors have modified the point-of-use water treatment system for use in disinfecting cabin air by including a reactive coating to interior of flow path, adapted interface points for aircraft environmental control system, optimized internal geometry to maximize contact time for airstream. The system 10 shown and described is generally constructed as a self-contained unit to be used as an air treatment system.

Referring now to the individual features of the system shown in FIG. 1, the reaction chamber 12 contains one or more germicidal UV light sources 14, which are typically housed inside a sleeve 16 (typically a quartz sleeve, but alternate sleeve options are within the scope of this invention and are described in more detail below). Air enters the treatment system 10 from a bottom inlet 18, flows through the chamber 12 as described below, and exits through top outlet 20, instantly ready for use. Inlet 18 and outlet 20 are generally tubular or circumferential in nature.

In one embodiment, inlet 18 is generally in fluid communication with an air source, such as an air tank or cabin duct air, and is configured to deliver air to the chamber 12. Outlet 20 extends out of the chamber 12, and its free end is in fluid communication with an air-dispensing apparatus, such as a vent to deliver air to the end user or an inlet valve to deliver air to a proton exchange membrane fuel cell. For example, the system 10 may be placed in the air ducts of the cabin and the air can be purified either actively or passively. Similarly, the device can be placed into the inlet tube for the cabin air into the PEMFC with either the active or passive method as well.

In the embodiment shown in FIG. 2, air entering the chamber 12 is directed through an air flow space 22 positioned generally in the center of the chamber 12. Air flow space 22 is typically an annular tube, although it should be understood that any appropriate channel or configuration may be provided. In this example, a UV light source 14 is generally positioned within the air flow space 22 in order to treat the air. The UV light source 14 positioned inside the air flow space 22 is generally protected by a sleeve 16 (alternate options for which are described below). The air to be treated enters the air flow space 22, around the outside of the UV source 14 and sleeve 16. The interior of the chamber wall 12 is covered with the PCO coating 13 or the PHFREME equivalent in order to produce radical oxidative species when contacted with the UV light.

Alternatively, in the embodiment shown in FIG. 3, air entering the chamber 12 is directed through an air flow space 22 positioned generally in the center of the chamber 12, and one or two (or more) UV light sources 14 may be positioned alongside each side of the air flow space 22 within the chamber 12, in order to treat the air. Although two UV light sources 14 are shown and described, it should be understood that only a single UV source or more than two sources may be used. The interior of the chamber wall 42 should be covered (at least partially, and in some instances, substantially fully coated) with the PCO coating or the PHI/REME equivalent in order to produce radical oxidative species when contacted with the UV light.

In either of these reaction chamber embodiments, one or more reflectors 24 may be positioned near or against the chamber wall 26 to help light reflection and enhance treatment. The reflector 24 may be a removable sleeve or liner inside the chamber 12, and may be made of any appropriate reflective material, whether metal or non-metal. For example, exemplary reflectors may be made of ceramics or polymers, or may have coatings of ceramics or polymers, or specifically, may have polymeric coatings with a high gloss finish. Alternate reflectors may be anodized aluminum-based, with or without the described coatings. In one embodiment, there may be provided an anodized coating that may have at least a portion stripped away in order to obtain conductivity and ground the unit for safety. In other words, the reflector can be etched to establish a conductivity point. Even in the instance where a high gloss finish is used, there may be an etched portion to establish a contact point. The general intent for the reflectors is to provide as much reflection of the UV light back into the system as possible.

UV light sources 14 are typically referred to as UV lamps. The UV lamps are typically enclosed by sleeves 16 to protect the lamps and help reflect light. The UV light source 14 may be any appropriate UV light source, such as low or high pressure UV lamps, standard UV bulbs, or light-emitting diode (LED) sources, as described herein. The light is mounted so that as air passes through a flow chamber, UV rays are admitted and absorbed into the air stream. When UV energy is absorbed by the reproductive mechanisms of bacteria and viruses, the genetic material (DNA/RNA) is rearranged so that they can no longer reproduce, killing the bacteria and eliminating the risk of disease. UV treatment thus disinfects air without adding disinfection chemicals.

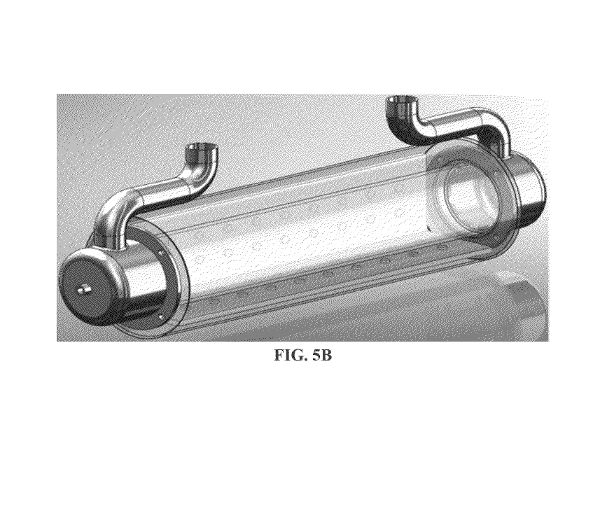

In a preferred embodiment, the UV light source is provided as one or more light emitting diodes (LEDs) 30 that are positioned anywhere in the reaction chamber, as long as they are able to emit light having a disinfection wavelength to the air being treated. This may also be an array of LEDs with clusters emitting at different wavelengths so as to create a spectrum effect. The LEDs may either be positioned inside the air flow (in a configuration similar to that shown in FIG. 2) or positioned outside the air flow (as shown in FIGS. 3, 5A, and 5B). The general goal is to expose the air in the flow passage to the UV LED wavelength. Any geometry that allows the LEDs to be arranged around or in the air is considered within the scope of this invention.

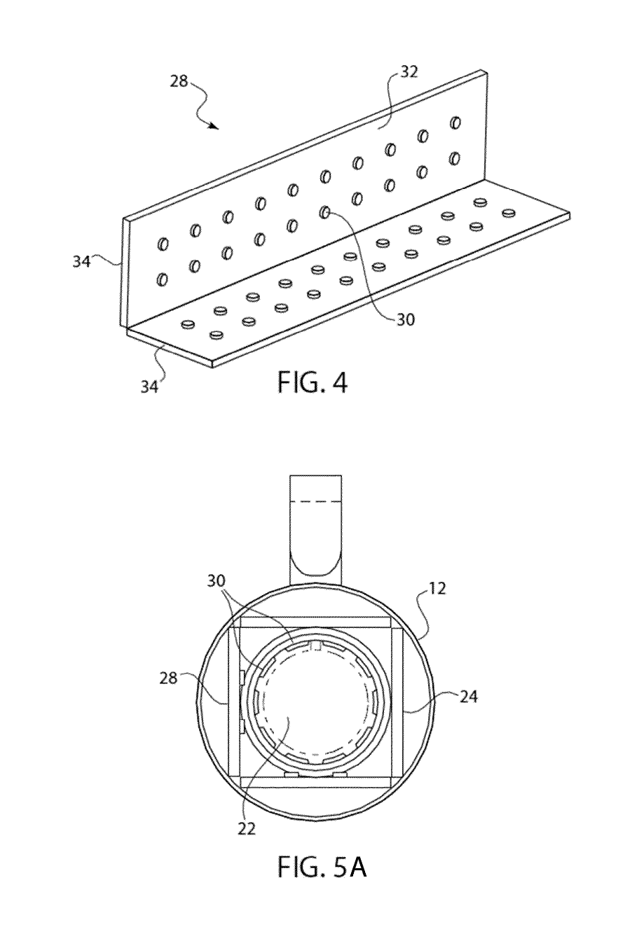

For example, as shown in FIG. 5A, the UV light source may be provided as a set of one or more light emitting diode (LED) units 28. One example of an LED unit 28 is shown in FIG. 4. The LEDs 30 themselves are manufactured to emit light in the ultraviolet range. They may be provided as individual LEDs 30 arranged in various positions directly on the chamber wall 26, on or against air flow space 22, or they may be arranged on units 28 as shown. If arranged on units 28, one or more sides of the unit 28 may be provided with a reflective surface 32 in order to help reflect to the UV light emitted more effectively.

The UV LEDs may be positioned in any desired configuration. One example is the box-shaped configuration shown in FIG. 5A, which is formed by two L-shaped units of FIG. 4. In the L-shaped embodiment shown, one or both panels 34 may be provided with a reflective surface 32. In one specific use, the units 28 may be positioned around the air flow space 22 at angles to one another so that a box-shaped unit is formed.

Alternate configurations are possible and within the scope of this invention. For example, although an L-shaped LED unit 28 and a box-shaped configuration are shown, it should be understood that any appropriately shaped unit may be used and is considered within the scope of this invention. For example, the unit 28 may be provided as a cylindrical or partially cylindrical unit (e.g., a tubular unit, a circular, round or oval unit, or a half circle unit, two partially separated halves), a square or rectangular unit, single panels, a 3-sided triangular unit, a straight or curved configuration, or any other appropriately shaped unit. Moreover, although the LEDs 30 are shown as being provided in two rows in FIG. 4, it should be understood that fewer or more rows may be provided or that the LEDs may alternatively be scattered in random patterns along one or more panels 34, along the inside wall of chamber, along the air flow space 22, or in any other appropriate position in chamber 12, as long as the LEDs are able to treat the air in the system.

In the specific embodiment shown, the design of the unit has an L-shape array of UV LEDs and a corresponding L-shaped reflector that emits UV light and reflects on to the air to be treated. The UV LED and reflector units may be used as structural components. This arrangement allows for the use of a larger quartz sleeve to maximize air flow rate. In other words, when a UV array is positioned on the outside of the air flow space, there is provided a larger passage for the air, which allows the air path to be larger, and as such, allows more air to be treated per pass.

An alternative UV LED arrangement with the array inset in the air flow/reaction chamber is diagrammed in FIG. 5B. In this embodiment, the LEDs are arranged on two flat panels, spaced to evenly illuminate a focal center. An alternative is to arrange LEDs on the concave side of a curved panel, and spaced to evenly illuminate a focal center, as shown in FIG. 5B. Curvature can be of such a design that it facilitates the focus of light from illuminating LEDs into the target media, although other LED configurations are possible and within the scope of this invention. The array of LEDs can be coated with a TiO₂ layer, or an alternative photocatalyst to produce a PCO reaction directly adjacent to the air flow. This provides a dual benefit of irradiating pathogens from a closer distance and providing a larger surface area for PCO to occur.

In one embodiment, inlet 18 is configured with at least one bend 36, curve, or portion having a non-linear dimension along its length in order to prevent line of sight to the UV light source contained within the reaction chamber 12. Outlet 20 is also configured with at least one bend 38, curve, or portion having a non-linear dimension along its length in order to prevent line of sight to the light source contained within the reaction chamber 12. The bends 36, 38 (or curves or non-linear portions) of this design are primarily intended to protect maintenance personnel or anyone else who may come into direct contact or otherwise have their eyes positioned at or near inlet 18 or the outlet 20 from being directly subjected to the UV light. The bends 36, 38 prevent the UV light source from being immediately viewable, causing the light to refract and take differing paths along the inlet and outlet portions.

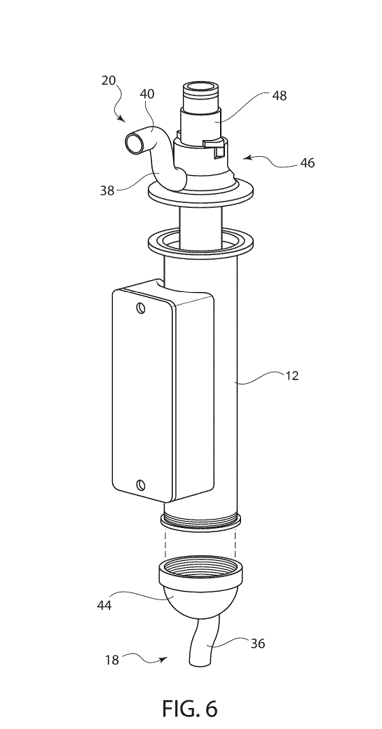

The system 10 is typically provided with a minimal number of components and in certain embodiments, has a modular construction, as shown in FIG. 6. A modular construction provides increased ease of maintenance and replacement. In one embodiment, the modular construction is provided by three main components: a removable lower cap 44 comprising an inlet 18, reaction chamber 12, and removable upper cap 46 comprising an outlet 20 and a lamp housing 48. However, it should be understood that the modular components may be provided alternatively as desired. The modularity provided by removable lower cap 44 and removable upper cap 46 allows easier cleaning, maintenance (e.g., disassembly and reassembly), and access to the UV lamp. The caps may be secured to the chamber 12 by any appropriate mechanism, such as threaded, bolted, clamped, or any other securing means.

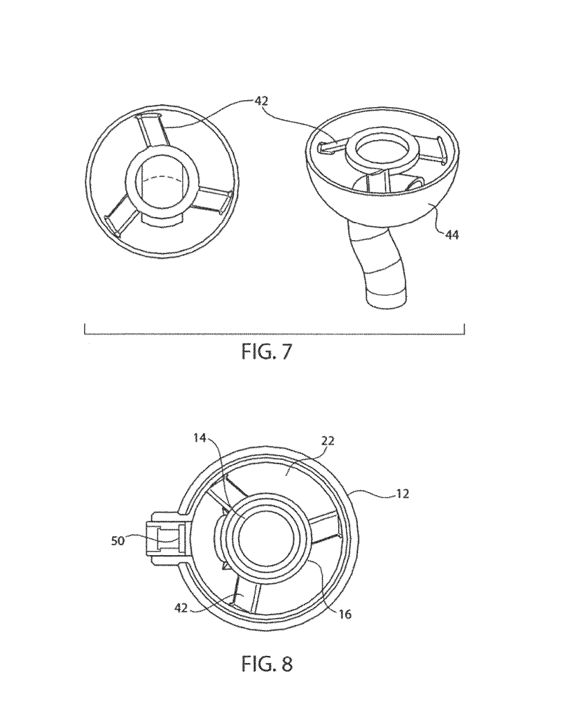

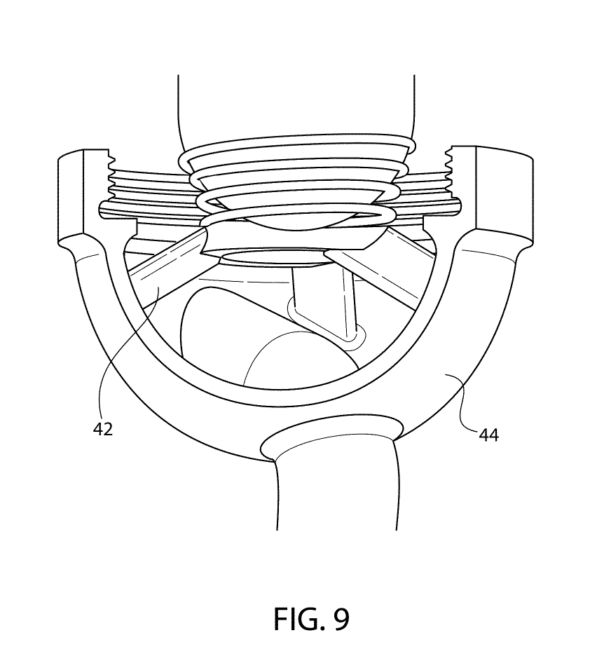

As shown in FIG. 7, air inlet 18 may have one or more turbulence inducing fins 42. In the specific embodiment shown, fins 42 are provided on the inside of lower cap portion 44, and are intended to introduce turbulence into the air entering the air flow space 22. Causing agitation of the air and creating a vortex helps ensure circulation of the molecules in the air and distribution of the UV light through all air in the system. The turbulence also keeps the air in the air flow space 22 for a longer time, allowing for a lengthened contact time. The fins 42 may serve the additional (or alternate) function of supporting a UV lamp. In one embodiment, fins 42 are secured to the sides of cap portion 44, as shown in FIGS. 8 and 9. In this feature, the fins 42 create turbulence while also supporting a UV light source, providing dual functions. In one embodiment, one or more springs may be associated with the tripod, typically at the tripod base, which absorb shock and support the free end of the sleeve and UV light source. In effect, the tripod/spring combination helps vibrationally isolate the UV light source by absorbing potential shock rather than transferring shock to the UV lamp.

The UV air disinfection system will be in communication with the environmental control system (ECS) to coordinate operation and conserve power. When the ECS is powered and operating at full capacity, such as during high-activity periods during flight or while on ground, the UV light source will be activated and air will be disinfected upon contact with the PCO coating in the unit before distribution to consumers or passengers. If the ECS is operating below full capacity, the UV air disinfection system will incorporate signals from the ECS and fine-tune UV light intensity to give the appropriate dose for disinfection. The system will be deactivated when the ECS is no longer operating, for example during aircraft shutdown overnight.

There may be features of certain HVAC systems that may require different control mechanisms for a treatment module. For example, if the module is running all the time, it may be possible to provide “once time” air passes as desired.

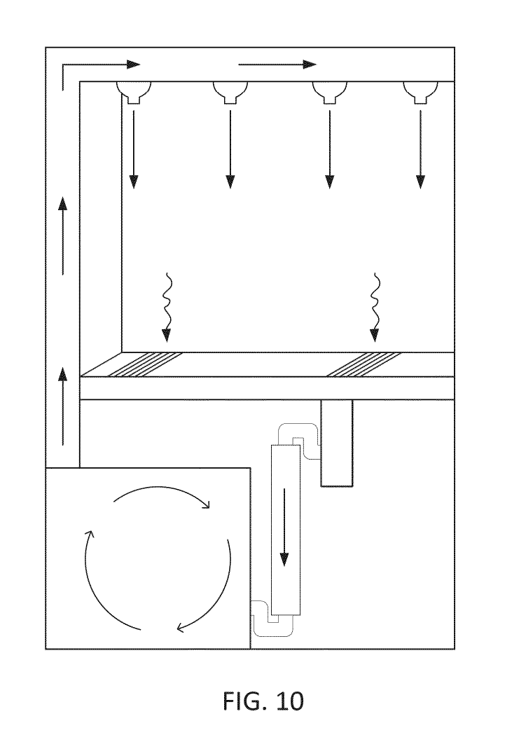

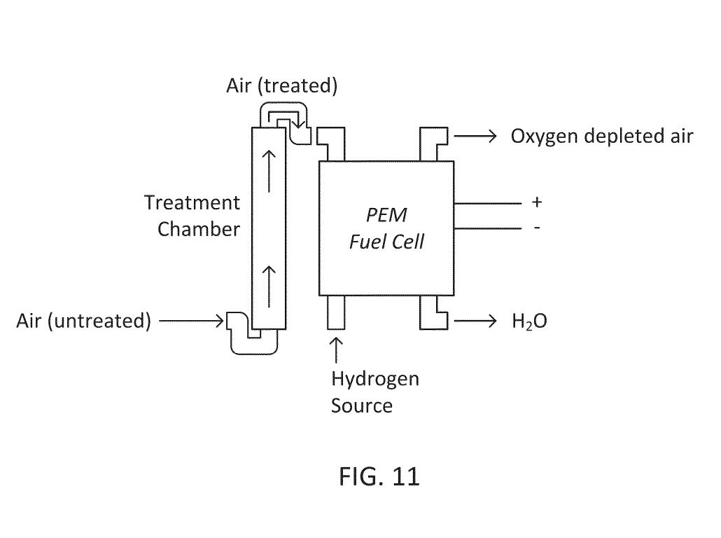

FIG. 10 shows an embodiment of this invention positioned in an air duct. Here, the device will emit radical oxidizers into the air stream which will then be carried by the inherent air flow in the duct to the target.

Changes and modifications, additions and deletions may be made to the structures and methods recited above and shown in the drawings without departing from the scope or spirit of the invention and the following claims. While the foregoing written description enables one of ordinary skill to make and use what is to be considered to be the best model thereof, those of ordinary skill will understand and appreciate the existence of variations, combinations, and equivalents of the specific embodiment, method, and examples herein.

What is claimed is:

1. An air treatment system, comprising:
(a) a reaction chamber comprising an inlet, an outlet, and an inner reaction surface;
(b) an air flow space positioned inside the reaction chamber;
(c) at least one or more ultraviolet light-emitting diodes positioned inside the reaction chamber;
(d) a photo-catalytic oxidative coating on the inner reaction chamber surface, wherein the inlet is configured to cooperate with an air inflow source and wherein the outlet is configured to cooperate with an air-dispensing apparatus.

2. The system of claim 1, wherein the inlet and the outlet each comprise a curved portion, and wherein the curved portion of the outlet comprises a first outlet bend nearest the reaction chamber and a second outlet bend at an outer end.

3. The system of claim 1, wherein the one or more ultraviolet light-emitting diodes are positioned within the air flow space.

4. The system of claim 1, wherein the one or more ultraviolet light-emitting diodes are positioned externally to the air flow space.

5. The system of claim 4, wherein the one or more light-emitting diodes are positioned on a unit external to the air flow space.

6. The system of claim 5, wherein the unit has one or more reflective surfaces.

7. The system of claim 5, wherein the unit is configured as a box-shaped unit.

8. The system of claim 1, wherein the system is modular.

9. The system of claim 1, wherein the system comprises one or more turbulence inducing fins.

10. The system of claim 1, wherein the air-dispensing apparatus comprises a vent to deliver treated air to an end user or an inlet valve to deliver treated air to a fuel cell.
